(19) United States
(12) Patent Application Publication
(10) Pub. No.: US 2006/0204449 A1
(43) Pub. Date: Sep. 14, 2006

(54) Nasal pharmaceutical composition of piribedil

(76) Inventors: Herve Rolland, Olivet (FR); Patrick Wuthrich, Orleans (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/564,139

(22) PCT Filed: Jul. 16, 2004
(86) PCT No.: PCT/FR04/01867

§ 371 Date: Jan. 10, 2006

(30) Foreign Application Priority Data
Jul. 17, 2003 (FR) 03/08712

(51) Int. Cl. A61K 31/724; A61K 31/506; A61K 9/14

(52) U.S. Cl. 424/46; 514/58; 514/252.2

(57) ABSTRACT

The present invention relates to a pharmaceutical composition for the administration, by the nasal route, of piribedil in solution or in powder form.

The present invention relates to a pharmaceutical composition for the nasal administration of piribedil.

Piribedil is a dopamine agonist which stimulates dopamine receptors and the cerebral and peripheral dopaminergic pathways.

Piribedil has hitherto been administered by the oral route in the form of prolonged-release tablets to be swallowed with half a glass of water. The said piribedil tablets are useful in the treatment of chronic pathological cognitive and neurosensory deficit in the elderly patient, in the ancillary treatment of intermittent claudication in chronic occlusive arteriopathies in the lower limbs and in the treatment of Parkinson's disease.

Piribedil may also be administered by the injectable route in order to improve the painful manifestations of arteriopathies in ischaemic attack, sometimes in association with surgical treatment.

Pharmacokinetic studies in humans have shown that the bioavailability of piribedil by the oral route is low in relation to the parenteral route and is subject to considerable variation within one and the same individual and from one individual to another.

The currently marketed form of piribedil is a prolonged-release form allowing gradual absorption and release of the active ingredient. Kinetic studies in humans have shown that, for the 50 mg dose, therapeutic levels are spread out over a period lasting more than 24 hours.

However, especially for the treatment of Parkinson's disease, the low bioavailability of piribedil and the inter- and intra-individual variations in concentration have resulted in the search for a new formulation allowing those problems to be solved. In addition, it was especially desirable for such Parkinson's patients that a rapid-action form be made available to medical staff in order to treat the very frequent acute attacks in those patients, especially for the rapid alleviation of akinesia.

The pharmaceutical compositions of the present invention make it possible not only to solve the known problems of the prolonged-release form but also to offer a superior medical service which especially allows the quality of life of patients to be improved. Being highly vascularised, the nasal mucosa is especially well suited to the rapid absorption of piribedil provided that the pharmaceutical form is matched to the characteristics of this active ingredient.

More especially, the pharmaceutical compositions according to the invention are characterised in that they comprise piribedil or a pharmaceutically acceptable salt thereof, optionally a cyclodextrin, and one or more pharmaceutically acceptable excipients.

The pharmaceutical compositions according to the invention are provided in the form of aqueous solutions or powders which can be administered to humans with the aid of a suitable device allowing the amount of piribedil that is required for obtaining the appropriate therapeutic effect to be delivered on each spray.

In the pharmaceutical compositions according to the invention, the piribedil is in the form of the base or a pharmaceutically acceptable salt.

The piribedil is preferably used in the form of the base.

The cyclodextrins that may be used in the pharmaceutical compositions according to the invention are, more specifically, β-cyclodextrins. Among the β-cyclodextrins there may be mentioned, without implying any limitation, methylated or partially methylated β-cyclodextrins, hydroxypropyl-β-cyclodextrin and sulphobutyl ether-β-cyclodextrin. Preferred cyclodextrins are partially and randomly methylated cyclodextrins. Partially and randomly methylated cyclodextrin is preferably cyclodextrin wherein the degree of substitution by methyl groups is around 1.7 (RAMEB). In nasal solutions, preference is given to the addition of cyclodextrins.

The amount of piribedil (equivalent of base) in the pharmaceutical compositions according to the invention that are solutions ranges from 10 to 500 mg, preferably from 100 to 400 mg and the amount of cyclodextrin ranges from 75 to 3750 mg, preferably from 750 to 3000 mg, for a final aqueous solution of 10 ml.

Preferably, for a final aqueous solution of 10 ml, the amount of piribedil (equivalent of base) is 100 mg and the amount of partially methylated cyclodextrin (RAMEB) is 750 mg.

The aqueous solutions may be rendered isotonic by the addition of sodium chloride, for example. The pH of the aqueous solutions is preferably adjusted to 6 by the addition of hydrochloric acid.

In the pharmaceutical compositions according to the invention that are powders, the amount of piribedil ranges from 0.1 to 20 mg, preferably from 1 to 10 mg, and the amount of cyclodextrin ranges from 7.5 to 75 mg.

Clinical studies carried out in Parkinson's patients using the pharmaceutical compositions according to the invention have shown excellent tolerance in humans, better bioavailability and increased efficacy compared to the oral form currently marketed.

The following Examples illustrate the invention without limiting it in any way.

### EXAMPLE 1

Solution Formulation:

| Piribedil base | 100 | mg |
|---|---|---|
| RAMEB | 750 | mg |
| Sodium chloride | 68 | mg |
| 1N hydrochloric acid, q.s. | pH 6 | |
| Purified water, q.s. | 10 | ml |

This pharmaceutical composition is administered using a metering pump delivering 100 μl of solution, or 1 mg of piribedil base, on each spray.

### EXAMPLE 2

Solution Formulation:

| Piribedil base | 400 | mg |
|---|---|---|
| RAMEB | 3000 | mg |
| Sodium chloride | 65 | mg |
| 1N hydrochloric acid, q.s. | pH 6 | |
| Purified water, q.s. | 10 | ml |

This pharmaceutical composition is administered using a metering pump delivering 100 μl of solution, or 4 mg of piribedil base, on each spray.

### EXAMPLE 3

Powder Formulation:

| | |
|---|---|
| Piribedil base | 2 mg |
| RAMEB | 15 mg |
| Mannitol | 3 mg |

This pharmaceutical composition is administered using a powder spray delivering 20 mg of powder, or 2 mg of piribedil base, on each spray.

### EXAMPLE 4

Powder Formulation:

| | |
|---|---|
| Piribedil base, micronised | 10 mg |
| Mannitol | 5 mg |

This pharmaceutical composition is administered using a powder spray delivering 15 mg of powder, or 10 mg of piribedil base, on each spray.

### EXAMPLE 5

Powder Formulation:

| | |
|---|---|
| Piribedil monomethanesulphonate | 2.65 mg |
| Lactose | 17.35 mg |

This pharmaceutical composition is administered using a powder spray delivering 20 mg of powder, or 2 mg of piribedil base, on each spray.

### Clinical Studies

Kinetics, Tolerance and Bioavailability Study in Healthy Volunteers

A study was carried out in 24 healthy volunteers in order to assess the local tolerance of the pharmaceutical composition according to the invention and also the kinetics of the formulation.

This study was carried out using the formulation described in Example 1, administered with the aid of a metering pump which delivers 100 μl of solution on each spray. The doses of piribedil tested are as follows: 0.1 mg, 0.25 mg, 0.5 mg, 1 mg and 2 mg, administered using two sprays each of 100 μl.

By means of this test it has been possible to show that the local tolerance of the pharmaceutical composition according to the invention is very good up to the 2 mg dose. The results of the kinetics parameters have shown the following:
The maximum concentration (C max) at the 2 mg dose is about 14 ng/ml. This dose corresponds to the minimum effective plasma concentration found to have a therapeutic effect on the tremors of Parkinson's patients when the latter are treated by the injectable route.
The said maximum concentration is obtained 15 to 25 minutes after administration.
From these results it has been possible to deduce that the bioavailability of piribedil administered by the nasal route is from 50 to 70%.

1-6. (canceled)

7. A pharmaceutical composition in the form of an aqueous solution or powder for nasal administration of piribedil, comprising:
piribedil or a pharmaceutically acceptable salt thereof,
optionally a cyclodextrin, and
one or more pharmaceutically acceptable excipients.

8. A composition according to claim 7, wherein the piribedil is in the form of the base.

9. A composition according to claim 7, wherein the cyclodextrin is a partially methylated β-cyclodextrin.

10. A composition according to claim 9, wherein the cyclodextrin is a β-cyclodextrin wherein the degree of substitution by methyl groups is about 1.7.

11. A composition according to claim 7, wherein, for a final aqueous solution of 10 ml, the amount of piribedil is from 10 to 500 mg for an amount of cyclodextrin of from 75 to 3750 mg.

12. A composition according to claim 7, wherein the composition is in powder form and the amount of piribedil is from 0.1 mg to 20 mg for an amount of cyclodextrin of from 7.5 to 75 mg.
